(19) United States
(12) Patent Application Publication
(10) Pub. No.: US 2023/0207070 A1
(43) Pub. Date: Jun. 29, 2023

(54) METHOD FOR COMPUTING A FLOW OF AT LEAST ONE FIRST GAS EMITTED BY A SOURCE INTO THE ATMOSPHERE USING A SECOND TRACER GAS, AND ASSOCIATED PROCESS, SYSTEM AND KIT

(71) Applicants: Centre national de la recherche scientifique, PARIS (FR); UNIVERSITE DE REIMS CHAMPAGNE-ARDENNE, REIMS CEDEX (FR); TotalEnergies OneTech, COURBEVOIE (FR)

(72) Inventors: Ludovic DONNAT, LA MOTTE SERVOLEX (FR); Olivier DUCLAUX, EYZIN PINET (FR)

(73) Assignees: Centre national de la recherche scientifique, PARIS (FR); UNIVERSITE DE REIMS CHAMPAGNE-ARDENNE, REIMS CEDEX (FR); TotalEnergies OneTech, COURBEVOIE (FR)

(21) Appl. No.: 17/926,920

(22) PCT Filed: May 19, 2021
(86) PCT No.: PCT/EP2021/063325

§ 371 Date: Nov. 21, 2022

(30) Foreign Application Priority Data
May 20, 2020 (FR) FR2005345

(51) Int. Cl. G16C 20/30; G01N 33/00

(52) U.S. Cl. CPC G16C 20/30; G01N 33/0004; B64U 2101/00

(57) ABSTRACT

This method comprises:
obtaining first data representative of amounts of a first gas and second data representative of amounts of a second tracer gas emitted by the source together with the first gas,
calculating at least one coefficient of correlation between the amounts of the first gas and the amounts of the second gas from the first representative data and the second representative data;
obtaining a measured or computed flow of the second gas emitted by the source;
computing a flow of the first gas emitted by the source on the basis of the measured or computed flow of the second gas emitted by the source and the correlation coefficient.

### CROSS REFERENCE TO RELATED APPLICATIONS

The present application is a U.S. National Phase Application under 35 U.S.C. § 371 of International Patent Application No. PCT/EP2021/063325 filed May 19, 2021, which claims priority of French Patent Application No. 20 05345 filed May 20, 2020. The entire contents of which are hereby incorporated by reference.

### FIELD OF THE INVENTION

The present invention relates to a method for computing a flow of at least one first gas emitted by a source into the atmosphere, implemented by a computing system, comprising the following steps:
obtaining first data representative of amounts of first gas measured in the atmosphere at a distance from the source along a trajectory.

The first gas is in particular a greenhouse gas emitted by the source during a chemical reaction. The chemical reaction is for example a combustion reaction of a methane flow, producing in particular carbon dioxide. The first gas of which the flow is computed is for example the residual methane which has not undergone combustion.

### BACKGROUND

Preoccupations regarding protection of the environment have contributed to reinforcement of legislation on polluting emissions, especially in Europe.

Thus, industrial units, such as present in the petroleum or chemical industry, must adapt to increasingly demanding environmental constraints.

In particular, greenhouse gases are emitted during operations of extracting, transporting, refining and storing hydrocarbons. These emissions are tracked by operators and are regularly subject to reduction measures.

It is in particular necessary to characterize the sources of these greenhouse gases and the amounts of greenhouse gases emitted by these sources, with a view to ensuring that they are controlled and to reporting progress made.

However, the techniques used to identify sources of greenhouse gases and quantify diffuse and short-lived emissions are still not entirely satisfactory.

Specifically, these emissions are very difficult to measure, because they are often unchannelled, and potentially located close to pools or lakes or inaccessible locations, for example at height or at the centre of the unit in question.

One major difficulty in evaluating the emissions of a point source within an installation is often the difficulty or even the inability to get as close as possible to the source in order to measure the flow of gas emitted by the source into the atmosphere.

In addition, the concentration of certain emitted gases is low in the atmosphere, in particular in the case of combustion residue. It is therefore often difficult to measure sufficiently accurately and then quantify the flows of emitted gas, in view of the measurement inaccuracy.

Nonetheless, monitoring the quantity of gases emitted by the source at low concentrations is often useful, in particular to fulfil legal obligations, for safety reasons and/or in order to optimise the operation of the installation.

### SUMMARY

An object of the invention is therefore to provide a method allowing computing of the flow of at least one first gas emitted by a source, in particular when the first gas has a reduced concentration in the atmosphere, the method being simple to implement while being precise.

To this end, an object of the invention is a method of the abovementioned type, characterized by the following steps:
obtaining second data representative of amounts of a second tracer gas emitted by the source together with the first gas, the second representative data being measured in the atmosphere at a distance from the source along the trajectory;
calculating at least one coefficient of correlation between the amounts of the first gas and the amounts of the second gas from the first representative data and the second representative data;
obtaining a measured or computed flow of the second gas emitted by the source;
computing a flow of the first gas emitted by the source on the basis of the measured or computed flow of the second gas emitted by the source and the correlation coefficient.

The method according to the invention may comprise one or more of the following features, alone or in any technically possible combination:
the correlation coefficient is a constant, representative of a ratio between the amounts of the first gas along the trajectory and the amounts of the second gas along the trajectory;
the calculation of the correlation coefficient comprises integration of the first representative data along at least part of the trajectory so as to obtain a first integrated overall amount, integration of the second representative data along at least a second part of the trajectory so as to obtain a second integrated overall amount, the correlation coefficient being calculated from the first integrated amount and the second integrated overall amount;
it comprises integration of the first representative data over a plurality of parts of the trajectory so as to obtain a first integrated overall amount corresponding to each part of the trajectory, then integration of the second representative data over the same plurality of parts of the trajectory; for each part of the trajectory, calculation of an amount ratio between the first integrated overall amount and the second integrated overall amount over the part of the trajectory, the correlation coefficient being calculated as the mean of the calculated amount ratios;
the trajectory comprises a plurality of parallel lines;
the flow of the first gas emitted by the source is also computed on the basis of the ratio between the molar mass of the first gas and the molar mass of the second gas;
the emissions of the first gas and second gas from the source result from a chemical reaction, in particular a combustion, wherein the measured or computed flow of the second gas is calculated from a material balance of the chemical reaction, in particular a combustion balance;
the second gas is produced by the chemical reaction, a third gas being produced together with the second gas by the chemical reaction, the method comprising obtaining third data representative of amounts of the third gas, the measured or computed flow of the second gas being calculated taking into account the third representative data;
the second gas is carbon dioxide;
the first gas is methane, benzene and/or a volatile organic compound;
the flow is computed using the equation Q1=C×M1/M2×Q2, where C is the correlation coefficient, M1 the molar mass of the first gas, M2 the molar mass of the second gas, and Q2 the measured or computed flow of the second gas emitted by the source.

Another object of the invention is a method for measuring emissions of a source into the atmosphere, comprising the following steps:
collecting first data representative of amounts of at least one first gas in the atmosphere at a distance from the source, by a drone along a trajectory;
simultaneous collection by the drone of second data representative of amounts of a second tracer gas emitted by the source together with the first gas, along the same trajectory;
transferring the collected first and second representative data to a computing system;
using the computing system to implement the computing method as defined above.

The method according to the invention may comprise one or more of the following features, alone or in any technically possible combination:
it comprises a preliminary step of determining a wind direction and/or a configuration of an emission plume downstream of the source, the drone being flown based on the predetermined plume configuration.

Another object of the invention is a system for computing a flow of at least one first gas emitted by a source into the atmosphere, comprising:
a data obtaining module able to obtain first data representative of amounts of a first gas measured in the atmosphere at a distance from the source along a trajectory, the system being characterized in that the data obtaining module is able to obtain second data representative of amounts of a second tracer gas emitted by the source together with the first gas, the second representative data being measured in the atmosphere at a distance from the source along the trajectory,
the system comprising:
a module for calculating at least one coefficient of correlation between the amounts of the first gas and the amounts of the second gas, from the first representative data and the second representative data;
a module for obtaining a measured or computed flow of the second gas emitted by the source;
a module for computing a flow of the first gas emitted by the source on the basis of the measured or computed flow of the second gas emitted by the source and the correlation coefficient.

Another object of the invention is a kit for measuring the emissions of a source into the atmosphere, comprising:
a drone, able to fly in the atmosphere at a distance from the source along a trajectory;
the drone being able to measure, along the trajectory, first data representative of amounts of at least one first gas emitted by the source and second data representative of amounts of at least one second tracer gas emitted by the source together with first gas;
a computing system as defined above, able to receive the first and second representative data measured by the drone.

### BRIEF DESCRIPTION OF THE DRAWINGS

The invention will be better understood on reading the following description, which is given merely by way of example, with reference to the appended drawings, in which:

FIG. 1 is a schematic view of a first emissions measurement kit according to the invention;

FIG. 2 is a view of a gas source within an installation, and of the plume emitted by the gas source;

FIG. 3 is a detailed view of the plume resulting from the source emitting in established wind conditions;

FIG. 4 is a view of the flight plan implemented by the drone of the kit from FIG. 1;

FIG. 5 is a view of the measurements of amounts of at least one first gas carried out on a horizontal line when implementing the flight plan from FIG. 4;

FIG. 6 is a view of the measurements of amounts of at least one second tracer gas carried out on a horizontal line when implementing the flight plan from FIG. 4;

FIG. 7 is a view similar to FIG. 3, in the case of an emission in low-wind conditions.

### DETAILED DESCRIPTION

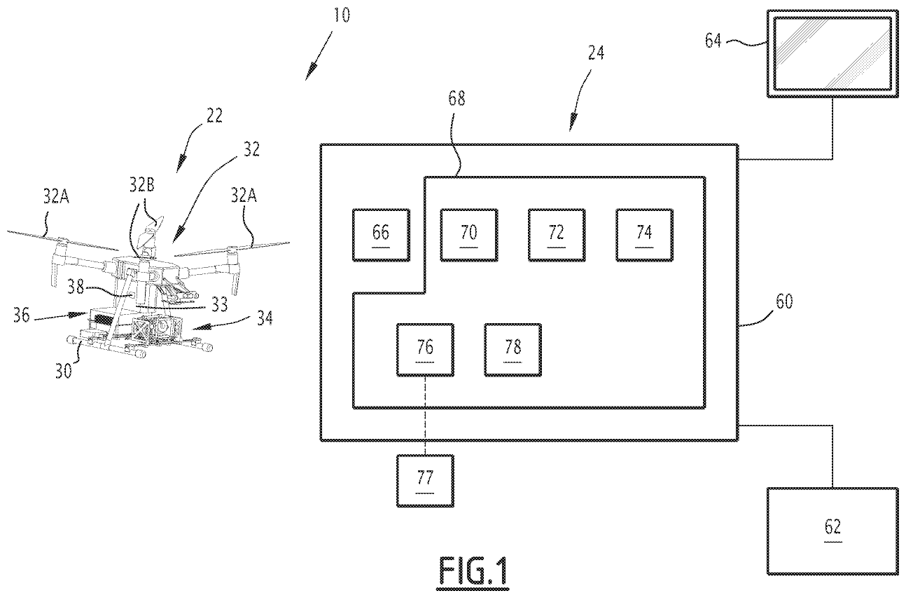

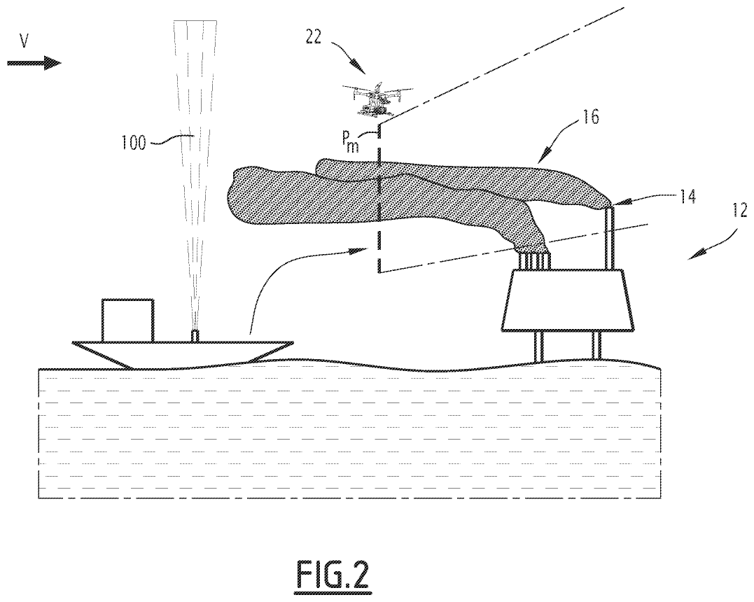

A kit 10 for measuring emissions of at least one first gas emitted by a source into the atmosphere, using a second tracer gas, is illustrated schematically in FIG. 1. The kit 10 is intended to implement a method for measuring emissions of an industrial installation 12, shown schematically in FIG. 2.

The installation 12 comprises at least one source 14 emitting the first gas, the amount of which is measured, and emitting the second tracer gas.

The first gas is preferably a gas with lower emissions than the second tracer gas.

The first gas and the second gas are for example emitted simultaneously by the source 14 as a result of a chemical reaction, in particular a combustion reaction.

For example, the second gas is a product of a chemical reaction using the first gas as reagent. The first gas is then a residual reagent which has not reacted during implementation of the chemical reaction producing the second gas.

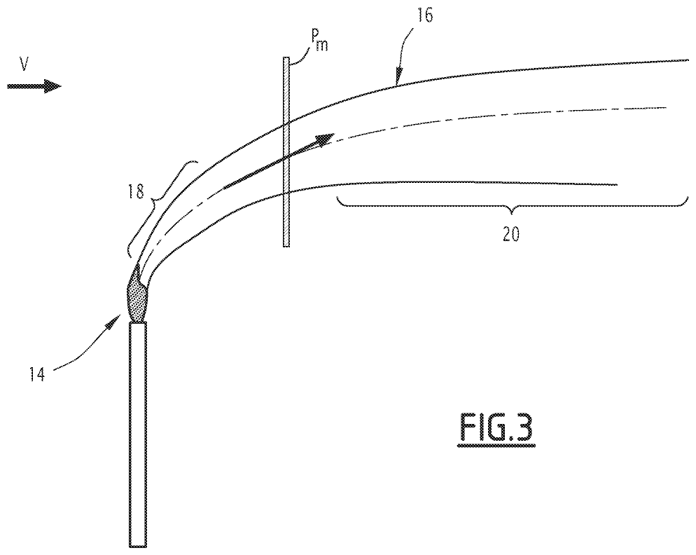

Advantageously, as illustrated on FIG. 3, the source 14 is a flare using combustion of a methane flow which constitutes the first gas. The second tracer gas is carbon dioxide produced by combustion of the methane within the flare.

In some variants, other gases may be measured, such as aromatic gases, especially benzene or even 1,3-butadiene, carbon monoxide, ethane and more generally volatile organic compounds.

The industrial installation 12 is in particular a petroleum installation, in particular a hydrocarbon extraction, transportation, refining, processing or storage installation located at sea or on land.

In the example shown in FIG. 3, the source 14 emits gases in a plume 16 which is released from the source 14 and propagates under the effect of the wind V.

The plume 16 is driven by the wind V blowing in the atmosphere close to the source 14. It advantageously has an area 18 in which the plume 16 rises, which is substantially vertical, and an area 20 in which the plume propagates, which is substantially horizontal in this example.

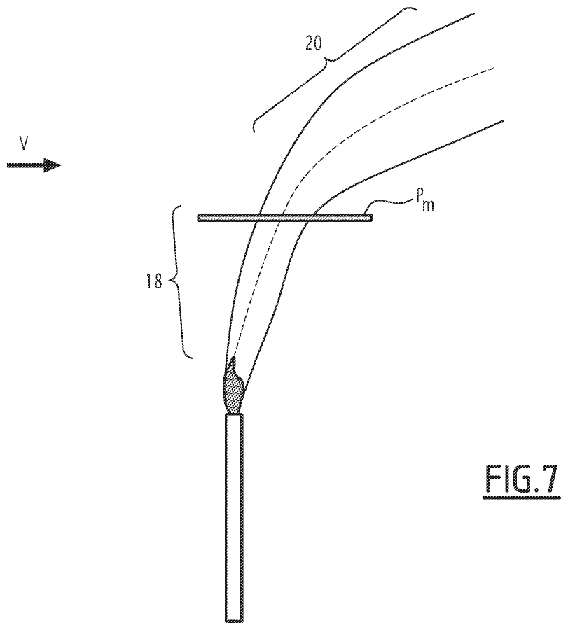

In the example of FIG. 7, if the wind V is lower, the rising area 18 is higher and the propagation area 20 extends in a manner inclined with respect to the horizontal.

The mean amount of first gas in the plume 16 is smaller than the mean amount of the second gas in the plume 16, for example by at least a factor of 10, or even at least a factor of 100.

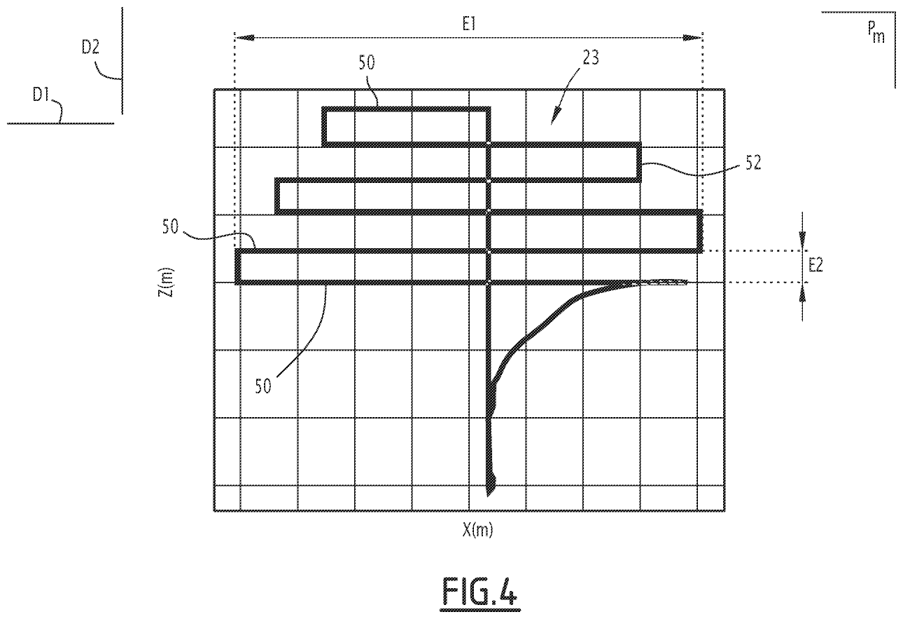

With reference to FIG. 1, to implement the measuring method, the measuring kit 10 comprises a drone 22 for collecting data representative of amounts of the first gas and second gas, at a plurality of positions in the atmosphere at a distance from the source 14 following a given trajectory 23, an example of which is shown in FIG. 4.

The kit 10 furthermore comprises a computing system 24, able to implement a method for computing a flow of the first gas emitted by the source 14 into the atmosphere, based on data representative of amounts of the first gas and second gas in the atmosphere as measured by the drone 22 along the trajectory 23.

The drone 22 is able to carry out the measurements needed to collect data representative of amounts of the first gas and second gas present in the plume 16, at a distance from the source 14. It comprises a chassis 30, and a propelling assembly 32, which is able to allow the chassis 30 to take off away from the ground and it to move by flying through the atmosphere above the ground.

The drone 22 furthermore comprises a measuring assembly 34, a control assembly 36 for controlling the measuring assembly 34, and preferably a remote transmission system 38.

With reference to FIG. 1, the propelling assembly 32 comprises a plurality of propelling members 32A, which here are propellers driven to rotate by a motor.

The propelling assembly 32 furthermore comprises a power source 32B, formed here by a battery, and a system 33 for locating and for controlling the movement of the drone 22 through the atmosphere.

In this example, the drone 22 is a multi-rotor rotary-wing drone. It does not have any fixed wings, its lift being generated by the propelling assembly 32.

The drone 22 is for example a rotary-wing quadcopter drone, and especially a DJI M200 drone as sold by DJI.

The propelling assembly 32 comprises a plurality of propellers that rotate about substantially vertical axes. “Substantially vertical” is generally understood to mean that the axes of rotation of the propellers are inclined by less than 30° with respect to the vertical.

When the motors of the propellers are supplied with electric power by the battery, the propellers are driven to rotate about their axis, driving a downward flow of air.

The locating and control system 33 comprises a position sensor, especially a GPS and/or an inertial measurement unit. It furthermore comprises a control unit, which is able to control the movement of the drone 22 along the trajectory 23, pre-recorded before the flight and loaded into the system 33, or remotely and manually via a remote control.

The drone 22 is thus able to automatically follow the predefined trajectory 23 or, alternatively, to be controlled manually by an operator, in order to implement the predefined trajectory 23 established in a flight plan.

Preferably, in order to implement the measuring method, the drone 22 is able to perform a trajectory 23 following a ladder-shaped movement, as illustrated by FIG. 4.

The drone 22 moves along a plurality of lines 50 parallel to a first direction D1, with a connecting segment 52 between each pair of adjacent parallel lines 50. The connecting segment 52 follows a second direction D2 transverse to the first direction D1.

Here, the first direction D1 is a horizontal direction and the second direction D2 is a vertical direction.

In this example, all of the parallel lines 50 scanned by the drone 22 extend substantially in one and the same vertical measuring plane Pm.

The extent E1 of the lines 50 in the first direction D1 is chosen based on the width of the plume 16, in order to scan the entire plume 16. This extent E1 is generally greater than 20 m and is between 20 m and 500 m.

The distance between the lines 50 is defined by an extent E2 of the connecting segments 52 in the second direction. This extent E2 is for example greater than 1 m and in particular between 1 m and 50 m.

The measuring assembly 34 comprises sensors able to carry out measurements of data representative of amounts of the first gas and second gas present in the atmosphere, at a plurality of points along each line 50.

Preferably, the data representative of the amounts of the first gas and second gas are collected by the measuring assembly 34 along each line 50.

The measurements are carried out continuously along the line 50. The measurement frequency of data representative of each gas amount is for example greater than 1 Hz and in particular between 1 Hz and 100 Hz.

One example of a measuring assembly 34 is described in application no. 20 03027 from the Applicant, filed at the Institut National de la Propriété Industrielle in France on 27 Mar. 2020, entitled “Drone for measuring data representative of amounts of at least two gases present in the atmosphere away from the ground and associated measuring method”.

The control system 33 comprises a data collection unit that comprises at least one memory able to store the data representative of each amount of each gas, in association with the geographical position along each line 50.

The data collection unit is connected to the remote transmission system 38 in order to allow the data to be exported to the computing system 24 when the drone is flying or after the drone has flown.

The computing system 24 is located on the ground here. It comprises at least a computer 60 and a human-machine interface comprising a control member 62 such as a keyboard, a mouse and/or a touchscreen, the human-machine interface also comprising a display 64, in particular a screen.

The computer 60 comprises, as is known, at least a processor 66 and a memory 68 comprising software modules able to be executed by the processor 66 in order to carry out functions. As a variant, the computer 60 comprises programmable logic components or dedicated integrated circuits intended to carry out the functions of the modules that will be described below.

With reference to FIG. 1, the memory 68 contains a module 70 for obtaining and initially processing first data representative of amounts T1 of the first gas and second data representative of amounts T2 of the second gas along each line 50 of the trajectory 23.

The memory 68 also contains a module 72 for computing at least one coefficient C of correlation between the amounts T1 of the first gas and the amounts T2 of the second gas from the first representative data and the second representative data.

The memory 68 also contains a module 74 for obtaining a measured or computed flow Q2 of the second gas emitted by the source 14, and a module 76 for computing a flow Q1 of the first gas emitted by the source, on the basis of the measured or computed flow Q2 obtained by the obtaining module 74 and the correlation coefficient C.

The obtaining and processing module 70 is able to receive the first data representative of amounts T1 of the first gas and the second data representative of amounts T2 of the second gas, along each line 50, as measured by the drone 22 at each measurement point, in association with the geographical position X of the measurement point along the line 50, each line 50 constituting a part of the trajectory 23.

It is able to transform the measured representative data into amounts T1, T2 of each of the gases, at each measurement point X on each line 50, on the basis of a calibration curve associated with each gas.

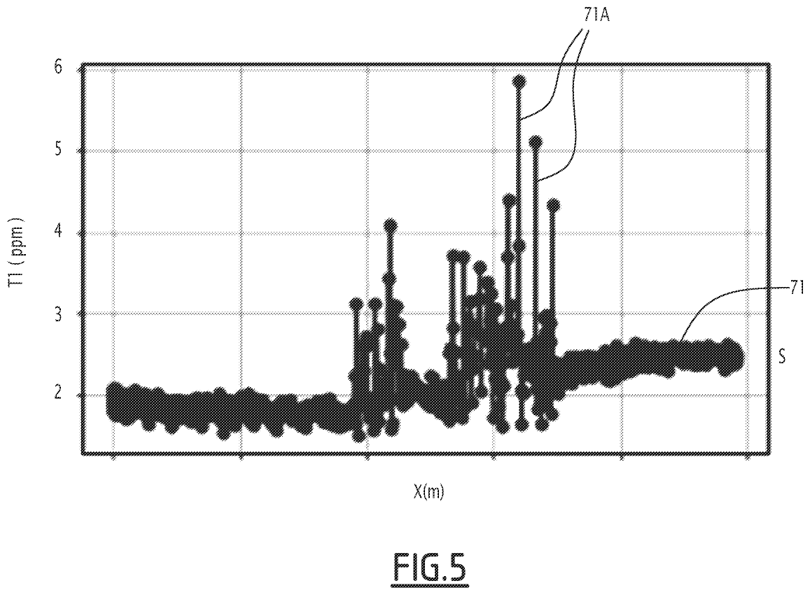

A curve 71 of amounts T1 of the first gas as a function of a first coordinate X along the line 50 in the direction D1 is thus obtained, as illustrated in FIG. 5.

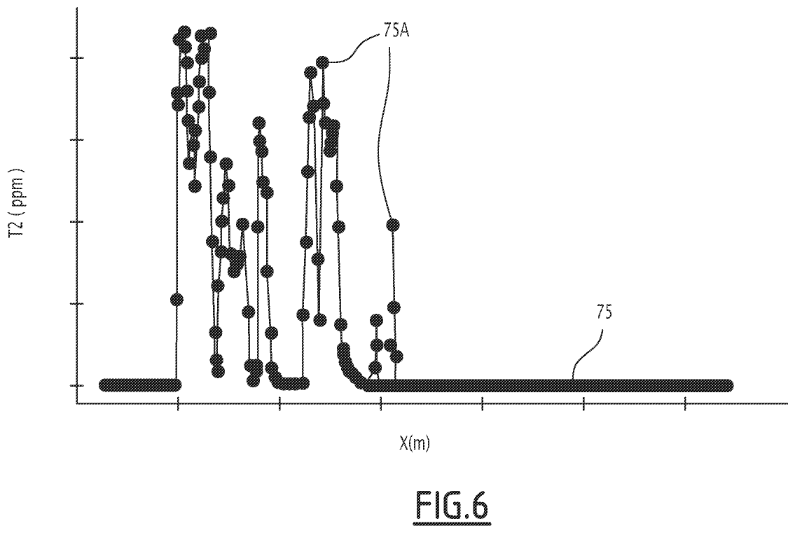

A curve 75 of amounts T2 of the second gas as a function of a first coordinate X along the line 50 in the direction D1 is thus obtained, as illustrated in FIG. 6.

The obtaining and processing module 70 may furthermore also be able to filter the obtained amounts.

According to a first method, the obtaining and processing module 70 is able to detect amount peaks 71A, 75A on each curve 71, 75 on the basis of a predetermined threshold S for the occurrence of a peak, and then to eliminate the observed peaks 71A, 75A from the obtained curve in order to obtain a curve of background values as a function of the first coordinate X.

In one variant, the obtaining and processing module 70 is able to implement an iterative algorithm in which the average value of the amounts along the line 50 is computed, and then in which all of the amounts above the average value are eliminated from the curve 71, 75, and then to repeat the steps of computing the average value and of subtracting amounts above the average value until a convergence criterion is met.

The convergence criterion is for example that the difference between the successive average values between two iterations is less than a predetermined value, for example less than 10%.

A continuous background is thus determined and is subtracted from the curve 71, 75 representing the amounts T as a function of the position X on each line 50.

The computing module 72 is able to integrate each curve 71, 75 representing the amounts of each gas along each line 50, in the first direction D1, over the entire width of the line 50 in order to obtain an integrated overall amount TGI1, TGI2 on each line 50, using the following equations:

TGI1=∫_{Xmin}^{Xmax}T1(X)dX

TGI2=∫_{Xmin}^{Xmax}T2(X)dX

where X min and X max are the geographical coordinates characterizing the limits of the plume as defined along the extent E1 of the plume parallel to the first direction D1.

According to the first data processing method performed by the module 70, the integral of the curve of the background values is also computed and is subtracted from the previous integral.

According to the second method, the curve of background values is subtracted from the curve 70 of the amounts before integration.

Thus, for each line 50 in which a measurement has taken place, corresponding to a coordinate Z in the second direction D2, an integrated overall amount TGI1 (Z), TGI2 (Z) corresponding to each gas are obtained.

The computing module 72 is also able to calculate, for each line 50 in which a measurement has taken place, corresponding to a coordinate Z in a second direction D2, the ratio R(Z) of the integrated overall amount TGI1 (Z) of the first gas to the integrated overall amount TGI2 (Z) of the second gas.

The computing module 72 is also able to calculate the correlation coefficient C as being equal to the mean, preferably arithmetic, of the amount ratios R(Z) obtained on each line 50 defining part of the trajectory 23.

A correlation coefficient C thus corresponds to the mean ratio of the amounts of the first gas to the amounts of the second gas on each of the parts of the trajectory 23.

The module 74 for obtaining a measured or computed flow of the second gas emitted by the source is able to obtain or compute a flow Q2 of the second gas emitted by the source 14 either from measurement of the flow of the second gas at the source 14 or preferably from calculating the emissions of the second gas by the source when the source 14 emits the second gas during a chemical reaction.

The calculation is preferably obtained using a material balance on the basis of the flows of the reagents supplied to perform the chemical reaction, assuming a predefined efficiency of the chemical reaction.

Preferably, the chemical reaction is a combustion. The second gas is then carbon dioxide emitted by the source during combustion.

A combustion balance is then performed as a function of the flows supplied to the installation 12 to perform the combustion, taking into account a predefined combustion efficiency. Advantageously, the combustion balance is produced by ignoring the emissions of gases produced by the reaction other than the second gas, in particular the emissions of carbon monoxide in the case where the second gas is carbon dioxide.

The calculation by the material balance is carried out directly by the obtaining module 74 or is loaded from an external computing system 77.

Advantageously, the material balance, in particular the combustion balance, is produced using a calculation page of a data processing spreadsheet on the basis of the molar composition and the (volume or mass) flows of the reagents introduced into the reactor so as to perform the reaction.

A measured or computed mass flow Q2 of the second gas is thus obtained using the obtaining module 74.

The module 76 for computing the flow Q1 of the first gas emitted by the source is able to calculate the mass flow Q1 of the first gas emitted by the source, as a function of the mass flow Q2 measured or computed by the obtaining module 74, from the ratio of the molar mass M1 of the first gas to the molar mass M2 of the second gas, and from the correlation coefficient C, determined by the computing module 72 from the measurements of the amounts of the first gas and second gas performed by the drone 22.

Preferably, the first gas flow Q1 emitted by the source is obtained using the following equation:

Q1=C×M1/M2×Q2

If required, this calculation is repeated for a plurality of flights of the drone 22, each corresponding to a separate measurement campaign performed during emission by the source 14, then the flow Q1 of the first gas emitted by the source 14 is averaged over all flights performed.

A measuring method will now be described. Initially, the drone 22 is put into flight in order to take a trajectory 23 following a ladder-shaped movement in a measuring plane Pm, as illustrated by FIG. 4.

As indicated above, the drone 22 moves along a plurality of lines 50 parallel to a first direction D1, constituting parts of the trajectory 23, with a connecting segment 52 between each pair of adjacent parallel lines 50, the connecting segment 52 following a second direction D2 transverse to the first direction D1.

The first data representative of amounts T1 of the first gas and the second data representative of amounts T2 of the second gas are collected by the measuring assembly 34 along each line 50.

The measurements are carried out continuously along the line 50.

The memory of the data collection unit stores the data representative of each amount T1, T2 of each gas, in association with the geographical position X along each line 50.

Next, while the drone 22 is flying or after the drone 22 has flown, the remote transmission system 38 exports data to the computing system 24 on the ground.

The obtaining and processing module 70 receives the first data representative of amounts T1 of the first gas and the second data representative of amounts T2 of the second gas, along each line 50, as measured by the drone 22 at each measurement point, in association with the geographical position X of the measurement point along the line 50, each line 50 constituting a part of the trajectory 23.

It transforms the representative data into amounts T1, T2 of each of the gases at each measurement point X on each line 50 on the basis of a calibration curve associated with each gas. For each line 50, a curve 71, 75 of the amount of each gas as a function of a first coordinate X along the line 50 in the direction D1 is thus obtained, as may be seen in FIGS. 5 and 6.

The obtaining and processing module 70 may filter the obtained amounts, for example using the first method or the second method described above.

The integration module 72 then integrates each curve 71, 75 representing the amounts of each gas along each line 50, in the first direction D1, over the entire width of the line 50, in order to obtain an integrated overall amount TGI1, TGI2 on each line 50, using the following equations:

TGI1=∫_{Xmin}^{Xmax}T1(X)dX

TGI2=∫_{Xmin}^{Xmax}T2(X)dX

where X min and X max are the geographical coordinates characterizing the limits of the plume as defined along the extent E1 of the plume parallel to the first direction D1.

According to the first data processing method performed by the module 70, the integral of the curve of the background values is also computed and is subtracted from the previous integral.

According to the second method, the curve of background values is subtracted from the curve 70 of the amounts before integration.

Thus, for each line 50 in which a measurement has taken place, corresponding to a coordinate Z in the second direction D2, integrated overall amounts TGI1 (Z), TGI2 (Z) corresponding to each gas are obtained.

Then the computing module 72 calculates, for each line 50 in which a measurement has taken place, corresponding to a coordinate Z in a second direction D2, the ratio R(Z) of the integrated overall amount TGI1 (Z) of the first gas to the integrated overall amount TGI2 (Z) of the second gas.

The computing module 72 then calculates the correlation coefficient C as being equal to the mean, preferably arithmetic, of the amount ratios R(Z) obtained on each line 50 defining part of the trajectory 23.

The module 74 for obtaining a measured or computed flow of the second gas emitted by the source obtains or computes a flow Q2 of the second gas emitted by the source 14, either from measurement of the flow of the second gas at the source 14 or preferably from calculating the emission of the second gas by the source when the source 14 emits the second gas during a chemical reaction.

Then the module 76 for computing the flow Q1 of the first gas emitted by the source calculates the mass flow Q1 of the first gas emitted by the source, as a function of the mass flow Q2 measured or computed by the obtaining module 74, from the ratio of the molar mass M1 of the first gas to the molar mass M2 of the second gas, and from the correlation coefficient C determined by the computing module 72 from the measurements of the amounts of the first gas and second gas performed by the drone 22.

Preferably, the first gas flow Q1 emitted by the source is obtained using the following equation:

Q1=C×M1/M2×Q2.

As indicated above, this calculation may be repeated if necessary for a plurality of flights of the drone 22, each corresponding to a separate measurement campaign performed during emission from the source 14, then the flow Q1 of the first gas emitted by the source 14 is averaged over all flights performed.

The measuring method according to the invention is therefore particularly easy to implement, since it requires a simple measuring campaign using a drone 22 flying directly into the plume 16, at a distance from the source 14.

Following the measurement campaign, a simple and effective calculation may be made for obtaining a precise determination of the flow of a first gas emitted by the source 14 even if the measured amounts of the first gas in the plume 16 are low, thanks to the simultaneous measurement of amounts of a second tracer gas, the emitted flow of which can be measured or computed via the material balance.

This is particularly useful for detecting compounds in small amounts in the atmosphere.

For example, the table below illustrates the amounts of methane emitted by a source 14 constituted by a flare in an oil facility.

The total gas flow of the flare and the methane flow allow computation of the flow Q2 of carbon dioxide by performing a combustion balance.

The method according to the invention is used to measure a coefficient C of correlation between methane and carbon dioxide which is used to determine the flow Q1 of emitted methane from the flow Q2 of carbon dioxide calculated using the material balance. This allows determination, from a mean flare methane flow of 0.23 tonnes/hour, of a residual methane quantity equal to 0.7 g/h and hence an efficiency of 99% with respect to the methane destruction.

TABLE 1

| | | | Flow Q2 of | | |
|---|---|---|---|---|---|
| | | | carbon | | |
| | | | dioxide | | |
| | | | computed | | |
| | | Flow of | using the | | Flow Q1 of |
| | | supplied | combustion | | emitted |
| | Gas flow of | methane | balance | Correlation | methane |
| Flight | flare (t/h) | (t/h) | (g/s) | coefficient C | (g/s) |
| 1 | 5.6 | 0.49 | 2286 | 0.00078 | 0.7 |
| 2 | 5.3 | 0.42 | 2121 | 0.00073 | 0.6 |
| 3 | 5.3 | 0.37 | 2056 | 0.00044 | 0.3 |
| 4 | 5.6 | 0.26 | 2040 | 0.00120 | 0.9 |
| 5 | 5.4 | 0.22 | 1949 | 0.00225 | 1.6 |
| 6 | 5.2 | 0.13 | 1883 | 0.00095 | 0.7 |
| 7 | 5.3 | 0.13 | 1895 | 0.00083 | 0.6 |
| 8 | 5.4 | 0.13 | 1911 | 0.00122 | 0.9 |
| 9 | 5.6 | 0.13 | 1959 | 0.00076 | 0.5 |
| 10 | 5.6 | 0.12 | 1911 | 0.00081 | 0.6 |
| 11 | 5.4 | 0.12 | 1741 | 0.00145 | 0.9 |
| Mean | 5.4 | 0.23 | 1977 | 0.00104 | 0.7 |

The method according to the invention is able to be implemented close to various industrial installations 12, even if these installations are inaccessible or/and require safety precautions. The measurements may be performed at low cost and frequently, thereby making it possible especially to track the evolution of emissions brought about by the source 14, and to ensure that they are under control or that they are reduced.

In one variant, shown in FIG. 8, for low-wind conditions, the plume 16 has a vertical configuration and the measuring plane Pm is a horizontal plane. The first direction D1 is then a horizontal direction, and the second direction D2 is a horizontal direction perpendicular to the first direction D1. The measuring process, including the measuring method, remain similar to those described above.

Advantageously, the measuring method according to the invention comprises an initial step of determining structural characteristics of the plume 16, for example by measuring the wind rose applicable to the source 14 at the time of the measuring campaign.

In a variant, the amounts of carbon monoxide are measured in the plume 16 by the drone 22.

These amounts are integrated as described above, and an additional coefficient of correlation between the integrated overall amount of carbon monoxide and the integrated overall amount of carbon dioxide is obtained in order to correct the flow Q2 of carbon dioxide computed using the combustion balance.

In another variant, a correlation coefficient C(i) is calculated for each part of the trajectory 23, for example for each line 50. Then a flow Q1(i) of the first emitted gas corresponding to each part of the trajectory 23, in particular to each line 50, is obtained using the following equation:

Q1(i)=C(i)×M1/M2×Q2.

When the flow Q1 of the first gas emitted by the source is stable over time, it is possible to average, in particular by an arithmetic mean, the flows Q1(i) of the first emitted gas corresponding to each part of the trajectory 23, in particular to each part 50, in order to obtain a mean flow Q1.

However, when the flow Q1 of the first gas evolves over time, it is possible to follow the development of the flow Q1 over time by monitoring the different flows Q1(i) obtained over time for successive parts of the trajectory 23 or for successive flights.

In yet another variant, the trajectory followed by the drone 22 is different from the trajectory shown. For example, the trajectory comprises a plurality of curved and not straight lines (e.g. circle arc) parallel to one another, linked by connecting segments. This is advantageously the case when the lines are horizontal.

As another variant, the trajectory followed by the drone 22 comprises for example a volute trajectory or a zigzag, allowing the plume to be cut into different sections which may or may not be perpendicular to its main direction.

The method according to the invention is particularly effective for determining the flow of residual methane present in the plume 16 from a source 14 constituted by a flare in which the combustion of a methane flow is carried out. This is particularly the case when the second gas is carbon dioxide produced by the combustion of methane in the flare, the flow of which in the plume 16 is preferably computed using a combustion balance established on the basis of the flow of methane supplied to the flare.

1. A method to compute a flow of at least one first gas emitted by a source into the atmosphere, implemented by a computer, comprising:
obtaining first data representative of amounts of first gas measured in the atmosphere at a distance from the source along a trajectory,
obtaining second data representative of amounts of a second tracer gas emitted by the source together with the first gas, the second representative data being measured in the atmosphere at a distance from the source along the trajectory;
calculating at least one coefficient of correlation between the amounts of the first gas and the amounts of the second gas using the first representative data and the second representative data;
obtaining a measured or computed flow of the second gas emitted by the source;
computing a flow of the first gas emitted by the source using the measured or computed flow of the second gas emitted by the source and the correlation coefficient.

2. The method according to claim 1, wherein the correlation coefficient is a constant, representative of a ratio between the amounts of the first gas along the trajectory and the amounts of the second gas along the trajectory.

3. The method according to claim 2, wherein the calculation of the correlation coefficient comprises integrating the first representative data along at least part of the trajectory so as to obtain a first integrated overall amount, integrating the second representative data along at least a second part of the trajectory so as to obtain a second integrated overall amount, the correlation coefficient being calculated from the first integrated overall amount and the second integrated overall amount.

4. The method according to claim 3, comprising integrating the first representative data over a plurality of parts of the trajectory so as to obtain a first integrated overall amount corresponding to each part of the trajectory, then integrating the second representative data over the same plurality of parts of the trajectory; for each part of the trajectory, calculating an amount ratio between the first integrated overall amount and the second integrated overall amount over the part of the trajectory, the correlation coefficient being calculated as the mean of the calculated amount ratios.

5. The method according to claim 1, wherein the trajectory comprises a plurality of parallel lines.

6. The method according to claim 1, wherein the flow of the first gas emitted by the source is also computed using the ratio between the molar mass of the first gas and the molar mass of the second gas.

7. The method according to claim 1, wherein emissions of the first gas and second gas from the source result from a chemical reaction, the measured or computed flow of the second gas being calculated from a material balance of the chemical reaction.

8. The method according to claim 7, wherein the emissions of the first gas and second gas from the source result from a combustion, the measured or computed flow of the second gas being calculated from a combustion balance.

9. The method according to claim 8, wherein the source is a flare implementing a combustion of a methane flow, the second gas being carbon dioxide produced by the combustion methane within the flare.

10. The method according to claim 7, wherein the second gas is a product of the chemical reaction using the first gas as reagent, the first gas being a residual reagent which has not reacted during implementation of the chemical reaction producing the second gas.

11. The method according to claim 7, wherein the second gas is produced by the chemical reaction, a third gas being produced together with the second gas by the chemical reaction, the method comprising obtaining third data representative of amounts of the third gas, the measured or computed flow of the second gas being calculated using the third representative data.

12. The method according to claim 1, wherein the second gas is carbon dioxide.

13. The method according to claim 1, wherein the first gas is methane, benzene and/or a volatile organic compound.

14. The method according to claim 1, wherein the flow Q1 is computed using the equation Q1=C×M1/M2×Q2, where C is the correlation coefficient M1 the molar mass of the first gas, M2 the molar mass of the second gas, and Q2 the measured or computed flow of the second gas emitted by the source.

15. A method for measuring emissions of a source into the atmosphere, comprising:
collecting first data representative of amounts of at least one first gas in the atmosphere at a distance from the source, by a drone along a trajectory;
simultaneously collecting by the drone, second data representative of amounts of a second tracer gas emitted by the source together with the first gas, along the same trajectory;
transferring the collected first and second representative data to a computer;
the computer implementing the computing method according to claim 1.

16. The method according to claim 15, comprising preliminarily determining a wind direction and/or a configuration of an emission plume downstream of the source, the drone being flown based on the predetermined plume configuration.

17. A system to compute a flow of at least one gas emitted by a source into the atmosphere, comprising
a computer configured to obtain first data representative of amounts of a first gas measured in the atmosphere at a distance from the source along a trajectory, and configured to obtain second data representative of amounts of a second tracer gas emitted by the source together with the first gas, the second representative data being measured in the atmosphere at a distance from the source along the trajectory,
the
computer being configured to calculate at least one coefficient of correlation between the amounts of the first gas and the amounts of the second gas from the first representative data and the second representative data,
being configured to obtain a measured or computed flow of the second gas emitted by the source;
and being configured to compute a flow of the first gas emitted by the source using the measured or computed flow of the second gas emitted by the source and the correlation coefficient.

18. A kit to measure emissions of a source into the atmosphere, comprising:
a drone configured to fly in the atmosphere at a distance from the source along a trajectory;
the drone being configured to measure, along the trajectory, first data representative of amounts of at least one first gas emitted by the source and second data representative of amounts of at least one second tracer gas emitted by the source together with first gas;
a system according to claim 17, configured to receive the first and second representative data measured by the drone.
